(19) United States
(12) Patent Application Publication
(10) Pub. No.: US 2025/0090025 A1
(43) Pub. Date: Mar. 20, 2025

(54) ANALYZER SYSTEM AND METHOD FOR REAL-TIME SYNCHRONOUS DETECTION OF THE CHARACTERISTIC NEAR-INFRARED WAVELENGTH FEATURES OF OPTICALLY ACTIVE SUBSTANCES

(71) Applicants: DALHOUSIE UNIVERSITY, Halifax (CA); PROVINCIAL HEALTH SERVICES AUTHORITY, Vancouver (CA)

(72) Inventors: Ian Patrick Joseph ALWAYN, Oegstgeest (NL); Kevin Cecil HEWITT, Halifax (CA); Haishan ZENG, Vancouver (CA); Hao GUO, Halifax (CA); Alexey TIKHOMIROV, Halifax (CA)

(21) Appl. No.: 18/728,810

(22) PCT Filed: Jan. 20, 2023
(86) PCT No.: PCT/CA2023/050059

§ 371 Date: Jul. 12, 2024

Related U.S. Application Data
(60) Provisional application No. 63/301,239, filed on Jan. 20, 2022.

(51) Int. Cl. A61B 5/00; G01N 21/65; G01N 33/483

(52) U.S. Cl. CPC A61B 5/0075; A61B 5/4244; A61B 5/4872; G01N 21/65; G01N 33/4833; G01N 2201/06113

(57) ABSTRACT

An optical system and methods have been developed for real-time synchronous detection of vibration and/or rotation modes in biotic (e.g., fat, glyceride, vitamins, bilirubin, etc.) and abiotic systems (e.g., alcohol contents). The system and methods include a modulated light source (e.g., a CW laser of 1064 nm wavelength modulated at square wave signal at 5600 Hz by a square wave signal), laser beam shaping and light collecting optics, optical detectors, appropriately selected optical filters, mechanical or electronic laser beam modulator, electrical signal amplifiers (e.g., transimpedance, current and voltage amplifiers), synchronous detector (e.g. lock-in amplifier), data acquisition and hardware and software control systems. One or multiple lock-in amplifiers are used to extract weak signals from noisy background. The system has three configurations/embodiments for in-situ and ex-situ end uses—(i) tabletop probe, (ii) handheld probe and (iii) miniature handheld probe. The handheld probe is for ex-situ and open surgery whereas the tabletop probe can be combined with other systems for ex-situ (monitoring) assessments. The miniature handheld probe can be used in conjunction with needle biopsies. The weak signal of characteristic optical scattering (e.g., Raman scattering) peaks of target biotic indicators (e.g., glyceride, vitamins, bilirubin, etc.) and abiotic molecules (e.g., alcohol) are identified using sensitive lock-in amplification technique, which supersedes the state-of-the-art for other similar approaches and allows for the detection of weak Raman signals in ambient light conditions (e.g., LED and luminescent light). Without restricting the generality of the present disclosure, the system has been shown to provide a quantitative result of the fat content quickly and accurately in (i) lipid phantoms and (ii) liver samples, demonstrating a strong linear correlation (e.g., r>0.98) between output voltage signals and fat contents in the clinically relevant range.

### FIELD

The present disclosure is generally directed to an optical system and methods for real-time synchronous detection of vibration modes in biotic and abiotic systems, including but not limited to real-time steatosis and quantitative fat content determination.

### BACKGROUND

The obesity epidemic has resulted in an increased incidence of fatty liver disease in our population, including potential organ donors. M. Charlton. “Cirrhosis and liver failure in nonalcoholic fatty liver disease: Molehill or mountain?” Hepatology 47 (2008) 1431-1433. Y. F. Cheng, et al., “Assessment of donor fatty livers for liver transplantation” Transplantation 71 (2001) 1221-1225. When transplanted, fatty livers place patients at a higher risk for graft dysfunction and lower survival rates.

The gold standard to detect the amount of fat in the liver is with a liver biopsy, but unfortunately there is usually insufficient time to obtain and process biopsies during organ retrieval and subsequent transplantation. As such, many potentially viable donor livers are discarded based on a subjective assessment of fat content by the surgeon during organ retrieval; and conversely unviable ones may be transplanted. There are other applicable methods available for liver steatosis assessment, such as Magnetic resonance imaging (MRI), computational tomography (CT), and biochemical analysis. Each of these methods has their own standards for what is considered a normal liver, and at which point the liver is considered to be fatty or have non-alcoholic fatty liver disease (NAFLD). For histology a liver is considered to have normal amounts of fat if less than 5 percent of the hepatocytes (liver cells) in the sample contain macroscopic fat droplets: anything greater than this is considered to have fatty liver disease. E. Brunt. “Nonalcoholic Fatty Liver Disease: Pros and Cons of Histologic Systems of Evaluation” Int. J. Mol. Sci. 17 (2016) 97.

Other methods define a liver to be normal when the amount of fat in it is less than 5 percent by volume or by weight. Hoyumpa. A. M., Greene. H. L., Dunn. G. D. et al. “Fatty liver: Biochemical and clinical considerations” Digest Dis Sci 20, 1142-1170 (1975). Fabbrini E. Sullivan S. Klein S. “Obesity and nonalcoholic fatty liver disease: biochemical, metabolic, and clinical implications” Hepatology. 2010 February: 51 (2): 679-89.

MRI uses the natural magnetic properties in a sample (human, animal, phantom, etc.) to produce detailed images and spectra, based on Nuclear Magnetic resonance phenomenon. The hydrogen nucleus (single proton) is mainly used due to the large quantities of it in both water and fat. MRI is regarded as the most accurate non-invasive method to assess liver steatosis. It determines the fat signal fraction to estimate the liver triglyceride concentration. S. Meisamy et al., “Quantification of Hepatic Steatosis with T1-independent. T2*-corrected MR Imaging with Spectral Modeling of Fat: Blinded Comparison with MR Spectroscopy” Radiology 258 (2011) 767-775. Using this technique, livers with 5 percent of liver cells with macroscopic fat determined by histology correspond to a fat fraction of 6.4 percent. A. Tang et al., “Nonalcoholic Fatty Liver Disease: MR Imaging of Liver Proton Density Fat Fraction to Assess Hepatic Steatosis” Radiology 267 (2013) 422-431. Therefore, systems and methods that determine the fraction of macroscopic fat in liver cells should be able to detect as low as 5 percent of macroscopic fat content, as that is the cut-off for normal livers.

Raman spectroscopy is a non-destructive, inelastic light scattering technique that provides a spectral fingerprint by which molecules, such as triglycerides, can be identified. When light interacts with matter it gets scattered. There are multiple scattering effects that occurs, the most common being elastic Rayleigh scattering. In this process a photon excites an electron to a virtual state then the electron returns to the ground state and emits a photon of the same frequency as the incident photon. In ˜0.0000001% of scattering interactions the electron will fall to an excited vibration state, thus releasing a photon of lower energy therefore longer wavelength. This phenomenon is known as Stokes Raman scattering. An even more unlikely scattering effect is anti-Stokes Raman scattering. In this process, an electron is already in its excited vibrational state and after being excited to a virtual state it falls to the ground state, releasing a photon of higher energy and shorter wavelength. These vibrational states are specific to molecules and therefore so are the energy differences. For this reason, these processes can be used to identify functional groups. A. Kudelski. “Analytical applications of Raman spectroscopy” Talanta 76 (2008) 1-8. Accordingly. Raman Spectroscopy can rapidly detect the vibration state of C—H bonds in triglycerides and to provide a quantitative and qualitative assessment of fat in livers simply by collecting light scattered from the surface or internal parts (if combined with biopsy) of the liver. K. C. Hewitt et al., “Accurate assessment of liver steatosis in animal models using a high throughput Raman fiber optic probe” Analyst 140 (19) (2015) 6602-6609.

The above information is presented as background information only to assist with an understanding of the present disclosure. No assertion or admission is made as to whether any of the above, or anything else in the present disclosure, unless explicitly stated, might be applicable as prior art with regard to the present disclosure.

### SUMMARY

The present disclosure describes a system and method that allow transplant surgeons to obtain accurate measurements of liver fat content during donor surgeries. The measurement results are obtained rapidly and guide the surgeon in deciding whether the liver is safe to use for transplantation. This can lead to fewer discarded livers, reduced waitlists for liver transplantation and improved quality of life for many individuals with end-stage liver disease.

Disclosed herein is an optical system and methods for real-time synchronous detection of vibration and/or rotation modes in biotic (e.g., fat, glyceride, vitamins. bilirubin, etc.) and abiotic systems (for example, alcohol contents). The system may include a sample holder, incident light shaping optics, a collection system collecting the light scattered by the sample, informative channels selecting light spectral bands, photoelectric detectors converting scattered photons into electric signals, transimpedance, voltage and lock-in amplifiers amplifying and extracting informative signals, data acquisition and measurement control hardware and software systems, as well as data processing and analysis system which analyze informative signals and outputs the fat content of the sample.

Further provided herein are three example and non-limiting embodiments, which comprise different incident light shaping optics and light paths and scattering collection systems composed of different number of lenses, mirrors, optical pass filters, and optical fibers.

In an embodiment, the weak signal of optical characteristic peaks of target biotic indicators (e.g., glyceride, vitamins. bilirubin, etc.) and abiotic molecules (e.g., alcohol) are identified using sensitive lock-in amplification and/or current or voltage amplification techniques, which supersede the state-of-the-art for other similar approaches and allows for the collection of spectral information in the dark and ambient light (e.g., LED and luminescent light) conditions. Without restricting the generality of the present disclosure, the system can provide a quantitative result of the fat content quickly and accurately in (i) lipid phantoms and (ii) liver samples, demonstrating a strong linear correlation (e.g., r>0.98) between output voltage signals, and fat contents in the clinically relevant range. Without restricting the generality of the present disclosure, the system can provide a quantitative result of the fat content of human liver tissues, demonstrating a strong linear correlation (e.g., r>0.82) between the fat contents and the degree of steatosis in the clinically relevant range.

The foregoing summary provides some example aspects and features according to the present disclosure. It is not intended to be limiting in any way. For example, the summary is not necessarily meant to identify important or crucial features of the disclosure. Rather, it is merely meant to introduce some concepts according to the disclosure. Other aspects and features of the present disclosure are apparent to those ordinarily skilled in the art upon review of the following description of specific example embodiments in conjunction with the accompanying figures.

### BRIEF DESCRIPTION OF THE DRAWINGS

Embodiments of the present disclosure are described by way of example only, with reference to the attached figures, wherein:

FIG. 1 is a schematic block diagram of an example embodiment (a tabletop probe) in accordance with of the present disclosure.

FIG. 2 is a schematic block diagram of another example embodiment (a handheld probe) of the present disclosure.

FIG. 3 is a schematic block diagram of another example embodiment (a miniaturized handheld probe) of the present disclosure.

FIG. 4 provides an example of Raman spectra of fatty and normal rat livers measured using a FT-Raman spectrometer with 1064 nm excitation wavelength (Thermo Scientific Nicolet NXR 9650 with 1064 nm Nd:YVO₄ CW laser).

FIG. 5 provides an example of Raman spectra for 0%, 33%, 66%, and 100% sunflower oil phantoms (sunflower oil diluted in CCl₄) measured using a FT-Raman spectrometer with 1064 nm excitation wavelength.

FIG. 6 provides an example of Raman spectra for 0%, 33%, 66%, and 100% sunflower oil phantoms (sunflower oil diluted in CCl₄) in the high wavenumber region measured using a FT-Raman spectrometer with 1064 nm excitation wavelength.

FIG. 7 provides an example of the maximum recovered signal voltage obtained from embodiments of the present disclosure as a function of sunflower oil concentration in the sunflower oil phantoms (sunflower oil diluted in CCl4).

FIG. 8 provides an example of typical phase scan plots, in an embodiment, of the lock-in amplifier (recovered signal voltage vs phase difference between the recovered and reference signals) at different light modulation frequencies.

FIG. 9 provides an example of the maximum recovered signal voltage obtained from embodiments of the present disclosure as a function of the fat content in sunflower oil phantoms within the fat content range of 0-100%.

FIG. 10 provides an example of the maximum recovered signal voltage obtained from the embodiments of the present disclosure as a function of the fat content in pork lard phantoms for the clinically relevant fat content range.

FIG. 11 provides an example of the maximum recovered signal voltage obtained from embodiments of the present disclosure as a function of the fat content in duck fat phantoms within the fat content range of 3-100%.

FIG. 12 provides an example of the maximum recovered signal voltage obtained from the embodiments of the present disclosure as a function of triglyceride (TG) concentration for ex-situ rodent livers.

FIG. 13 provides an example of the differences of the maximum voltage signals of fat content measured using the embodiments of the present disclosure at ambient LED light conditions and in the dark for clinically relevant fat content range.

FIG. 14 provides an example of the correlation between the fat content in the sunflower oil phantoms and MRI signal within the fat content range of 1-75%.

FIG. 15 provides an example of the correlation between the fat content measured using embodiments of the present disclosure and one measured using MRI in sunflower oil phantoms.

FIG. 16 provides an example of the correlation between the fat content measured using one embodiment of the present disclosure and one measured using MRI in fatty duck liver samples.

FIG. 17 provides an example of the correlation between the fat content percentage of snap-freezing human liver specimens measured using one embodiment of the present disclosure and the degree of steatosis of the same specimens rated by pathologists.

FIG. 18 is a block diagram of an example computerized device or system.

The relative sizes and relative positions of elements in the drawings are not necessarily drawn to scale. For example, the shapes of various elements and angles are not necessarily drawn to scale, and some of these elements may be arbitrarily enlarged and/or positioned to improve the readability of the drawings. Further, the particular shapes of the elements as drawn are not necessarily intended to convey any information regarding the actual shape of the particular elements, and have been solely selected for ease of recognition in the drawings.

### DETAILED DESCRIPTION

An optical system and associated methods have been developed for real-time synchronous detection of vibration and/or vibration modes in biotic (e.g., fat, glyceride, vitamins. bilirubin, etc.) and abiotic systems (e.g., alcohol contents).

In some embodiments, the system includes a modulated light source (e.g., a 1064 nm CW laser), laser beam shaping and light collecting optics, optical detectors, appropriately selected optical filters, a mechanical or electronic laser beam modulator, transimpedance amplifiers, voltage and lock-in amplifiers, and data acquisition and measurement control hardware and software systems, as well as data processing and analysis systems. A lock-in amplifier is used to extract weak informative signals from a noisy background.

At least some of the embodiments of the present disclosure use Raman spectroscopy. Out of every million scattered photons approximately one is a Stokes Raman photon. In the Stokes Raman scattering process, a low energy photon is generated when the electron falls to an excited vibration state instead of ground state. The probability of Stokes Raman photon generation is 1:109. These vibrational states are specific to molecules and therefore so are the energy differences. Raman scattering can thus be used to identify optically active substances in a sample.

By detecting the intensity of different characteristic Raman scattering peaks, the present disclosure provides a quantitative result of target substances in samples simply by illuminating the surface of the samples with an infrared laser beam. Unlike conventional spectrometers, embodiments of the present disclosure do not need many technically demanding and expensive spectroscopic elements (e.g. gratings. CCD arrays etc.) in appointed tasks. The weak signal of optical characteristic peaks of target biotic indicators (e.g., glyceride, vitamins. bilirubin, etc.) and abiotic molecules (e.g., alcohol) are identified using a sensitive lock-in amplification technique that allows one to detect weak informative signal in the dark and in ambient light (e.g., LED light) conditions.

Without restricting the generality of the present disclosure, those quantitative results of the present disclosure reflect the fat content and the degree of steatosis in various samples. The present disclosure can provide transplant surgeons with quantitative and objective real-time results of the fat content in the liver by collecting light scattered by the surface or internal parts (if combined with biopsy) of the liver.

Without restricting the generality of the present disclosure, the present disclosure shows three configurations/embodiments for in-situ and ex-situ end uses-(i) tabletop probe. (ii) handheld probe and (iii) miniature handheld probe. The handheld probe is for ex-situ and open surgery whereas the tabletop probe can be combined with other systems for ex-situ (monitoring) assessments, and the miniature handheld probe can be used in conjunction with needle biopsies. The present disclosed invention has been shown to provide a quantitative result on the fat content quickly and accurately in (i) lipid phantoms and (ii) liver samples, demonstrating a strong linear correlation between intensities of output voltage signals. MRI readings, and fat contents in the clinically relevant range.

In an aspect, the present disclosure is directed to an optical system for real-time synchronous detection of vibration and/or rotation modes in biotic and abiotic systems, the system comprising: a light source, an incident light path, a sample stage, a scattering collection system, single or multiple informative channels, single or multiple current-to-voltage converters, single or multiple current or voltage amplifiers, single or multiple lock-in amplifiers and/or current or voltage amplifiers, photoelectric detectors, and a data analysis system, wherein a modulated light beam from the light source passes through the incident light path and excites the sample on the sample stage, and the collection system collects the scattered light emanating from the sample and focuses the light on the photoelectric detectors of the informative channels and the reference channel, and the current-to-voltage converter, current or voltage amplifier, and lock-in amplifiers amplify the output electric signals of the photoelectric detectors, and the data analysis system analyzes amplified signals collected by the channels and outputs the substance content of the sample.

In an embodiment, the data analysis system analyzes informative and reference signals extracted by the lock-in amplifiers and outputs the substance content of the sample.

In an embodiment, the lock-in amplifiers extract informative and reference signals directly or not directly from the pre-amplified signals output from the transimpedance amplifiers and/or voltage amplifiers.

In an embodiment, the system outputs a signal which is agnostic to ambient light conditions, including but are not limited to LED light conditions, or other means.

In an embodiment, the light source includes but is not limited to an infrared laser or other means alternative to lasers.

In an embodiment, the initial light is modulated in a periotic manner by an optical modulator or an electronic gating, or other means.

In an embodiment, the incident light path guides the output light beam from the laser source, shapes the beam, and alters the incident angle of the beam to a sample.

In an embodiment, the wavelength of the near infrared laser source and photoelectric detectors ranges from approximately 800 nm to approximately 1700 nm, and the optical elements are optimized for that wavelength range.

In an embodiment, the light passes through a combination of any number of optical elements including but not limited to lenses, mirrors, filters, beam splitters, or optical fibres.

In an embodiment, the photoelectric detectors, including but not limited to InGaAs photodiode detectors, convert optical signals into electric signals.

In an embodiment, one or multiple transimpedance amplifiers amplify electric signals directly or indirectly from the photoelectric detectors, including but not limited to informative, reference, background or noise signals.

In an embodiment, the combination of dichroic mirrors and optical pass filters selects specific optical wavelength bands of one or multiple informative channels.

In an embodiment, the samples include but not limited to in-situ and ex-situ liver organs or tissues.

In an aspect, the present disclosure is directed to a method of detecting vibration and/or rotation modes of a biotic or abiotic sample, comprising: placing a sample to be tested onto a test stage, illuminating the sample with a light beam to excite the sample, collecting scattered light from the illuminated sample and selecting characteristic optical bands with beam splitters and optical pass filters, converting light signals to electric signals with at least one photoelectric detector, converting current signals to voltage signals with current-to-voltage converters, extracting signals from noise with at least one lock-in amplifier and/or voltage amplifier, and calculating the amount of the substance content in the sample using the analysing system and the extracted voltage signals.

In an embodiment, the intensity of the extracted voltage signal of either channel represents the strength of characteristic vibration and/or rotation modes of the target substance.

In an aspect, the present disclosure is directed to an optical system for real-time synchronous detection of vibration and/or rotation modes in biotic and abiotic systems comprising a light source, an incident light path, a sample stage, a scattering collection system, single or multiple informative channels, single or multiple current-to-voltage converters, single or multiple lock-in amplifiers and/or operational amplifiers, photoelectric detectors, and a data analysis system, wherein a modulated light beam from the light source passes through the incident light path and excites the sample on the sample stage, and the scattering collection system collects the scattering light emanating from the sample and focuses the light on photoelectric detectors of the informative channels and a reference channel, and the current-to-voltage converter and lock-in amplifiers and/or operational amplifiers amplify the output electric signals of the photoelectric detectors, and the data analysis system analyzes amplified signals collected by the informative channels and outputs the substance content of the sample.

In an embodiment, the data analysis system analyzes informative signals extracted by the lock-in amplifiers and/or operational amplifiers and outputs the substance content results of the sample.

In an embodiment, the lock-in amplifiers and/or operational amplifiers extract informative signals directly or not directly from the pre-amplified signals output from the transimpedance amplifiers and/or operational amplifiers.

In an embodiment, the system outputs a signal which is unaffected by ambient light conditions, including but are not limited to LED shadowless light conditions in operating rooms, or other such means.

In an embodiment, the light source emits at least near-infrared light beams with a power of 500 mW or lower.

In an embodiment, the initial light is modulated in a periotic repetition rate by an optical modulator or an electronic gating, or other such means.

In an embodiment, the incident light path guides the output light beam from the laser source, expands the beam, and alters the incident angle of the beam to a sample.

In an embodiment, the wavelength of the near infrared laser source ranges from approximately 800 nm to approximately 1700 nm, and the optical elements are optimized for that wavelength range

In an embodiment, the light passes through a combination of any amount of optical elements including but not limited to lenses, mirrors, filters, beam splitters, or optical fibres.

In an embodiment, the photoelectric detectors, including but are not limited to InGaAs photodiode detectors, convert optical signals into electric signals.

In an embodiment, one or multiple transimpedance amplifiers amplify electric signals directly or indirectly from the photoelectric detectors, including but not limited to informative signals and noise signals.

In an embodiment, the combination of dichroic mirrors and optical pass filters selects specific optical wavelength bands of one or multiple informative channels.

In an embodiment, the samples include but are not limited to in-situ and ex-situ liver organs or tissues.

In an aspect, the present disclosure is directed to a method of detecting vibration and/or rotation modes of a biotic or abiotic sample, comprising the steps of placing a sample to be tested onto a test stage, illuminating the sample with a light beam to excite the sample, collecting scattered light from the illuminated sample and selecting characteristic optical bands with beam splitters and optical pass filters, converting light signals to electric signals with at least one photoelectric detector, converting current signals to voltage signals with current-to-voltage converters, extracting signals from noise with at least one lock-in amplifier and/or operational amplifier, and calculating the amount of the substance content results of the sample using the analysing system and the extracted voltage signals.

In an embodiment, the intensity of the extracted voltage signal of each channel represents the strength of characteristic vibration and/or rotation modes of the target substance, and the calculations of the substance content are based on common chemical knowledge.

### Definitions

The following paragraphs provide definitions of some of the terms used herein. All terms used herein, including those specifically described below in this section, are used in accordance with their ordinary meanings unless the context or definition indicates otherwise. Also, unless indicated otherwise, except within the claims the use of “or” includes “and” and vice-versa. Non-limiting terms are not to be construed as limiting unless expressly stated (for example. “including” is to be understood as meaning “including without limitation” unless expressly stated otherwise).

The “clinically relevant range” represents the fat content larger than 5% by volume fraction.

“Initial light” is the light emitted by a monochromatic light source.

“Incident light” or “excitation light” is the light used to irradiate the sample at a sample stage.

An “incident light path” is a light path from a light source to a sample. The path may comprise an optical modulator (mechanical chopper or an electronic gating), a coupling lens, a fiber optic cable, and a collimator and other beam shaping elements.

A “scattering collection system” is a combination of lenses, optical filters and mirrors through which light emanating from a sample is collected and focused on the photoelectric detectors. Light emanating from a sample includes light that being reflected by a sample, light transmitted through a sample, or light being generated within the sample. In the present application, such emanating light comprises Raman scatted light.

An “informative channel” is a combination of one or multiple optical filters and a photoelectric detector where the signal of a characteristic spectrum peak is the strongest and other signals are filtered.

A “data acquisition and analysis system” is a combination of a computer and necessary accessories, where the electric signals carrying information are acquired, digitized, and analyzed to output the content of target substances.

Other terms and phrases in this application are defined in accordance with the above definitions and in other portions of this application.

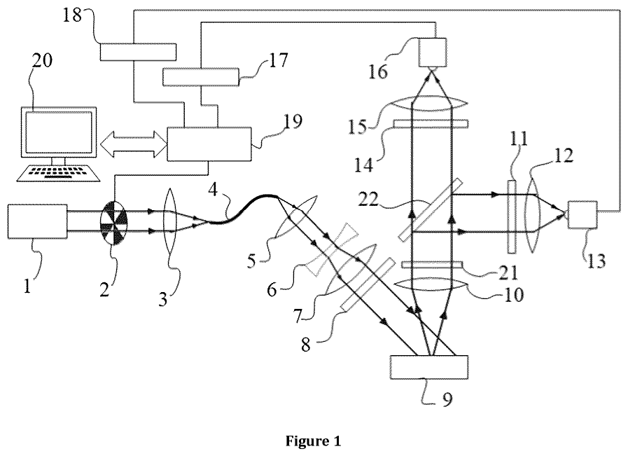

FIG. 1 depicts a schematic block diagram of a first embodiment of a system in accordance with the present disclosure. The embodiment in FIG. 1 includes an infrared light source 1 such as, for example, a 1064 nm continuous-wave Nd:YVO₄ laser. The initial light from the light source 1 is modulated by an optical modulator 2 (e.g. a chopper) and then converged by lens 3 and coupled into fiber optic cable 4 such as a Thorlabs M38L01 patch cable that transmits the initial light further down the incident light path. The fiber optic cable 4 is connected to a collimator, such as a Thorlabs F810SMA-1064 collimator, comprising lens 5, 6, and 7, to expand the beam of the initial light. The beam then goes through laser line filter 8 such as a Semrock LL01-1064-25 laser line filter to eliminate fiber emissions and pass only the laser excitation wavelength at 1064 nm. The incident beam excites samples disposed on sample stage 9. The samples are excited, and the scattered light from the samples is collected by a scattering collection system comprising lenses 10, 12, and 15. Optical filter 21 filters out Rayleigh scattered light, which has the same wavelength as the initial light. Beam splitter 22, for example, a Thorlabs DMSP1500 short pass dichroic mirror, splits the light between two informative channels. One of the informative channels comprises band-pass filter 11 such as a Chroma Technology ET1550/30 bp, lens 12, and photoelectric detector 13 such as a Thorlabs SM05PD5A InGaAs photodiode. The band-pass filter 11 passes the light of a spectrum peak at 1550 nm. The other informative channel comprises band-pass filter 14 such as a Thorlabs FL051064-10 laser line filter, lens 15, and photoelectric detector 16 such as a Thorlabs SM05PD5A InGaAs photodiode. The band-pass filter 14 passes the light of another spectrum peaks at 1064 nm, 1580 nm, 1620 nm, and so on. Transimpedance amplifiers 17 and 18 are used to convert the current signals produced by the photoelectric detectors 13 and 16 to amplified voltage signals. The voltage signals can go through a series of additional amplification stages (voltage amplifiers) and are detected by the lock-in amplifier 19 such as benchtop Stanford Research SR510 lock-in amplifier or any other type and model or an on-chip lock-in amplifier. The lock-in amplifier 19 uses the frequency signal of the optical modulator 2 as the reference signal. The lock-in amplifier 19 is connected, or otherwise intercommunicates, to the data acquisition and analysis system 20, which comprises an analog to digital converter, a computer, and data analysis software. The system may output the content of target substances in the samples on a computer display. The Raman photon collection efficiency of a typical informative channel, in an embodiment, is 1:2.25×10¹¹, which indicates that one Raman photon carrying critical information is collected when every 2.25×10¹¹ incident photons interact with the sample. As an example of the method for a real-time synchronous detection using this embodiment, the user places a sample to be tested onto the sample stage 9 and controls the start and/or the end of measurements manually or automatically via the analysis system 20. The incident light illuminates the sample, and light scattered from the illuminated area of the sample is collected by the light collection system. The user uses appropriate beam splitters and optical pass filters to select characteristic optical bands (in a typical case, in an embodiment. Raman characteristic peaks), and light signals of the selected characteristic optical bands are converted to electric signals with at least one photoelectric detector. At least one current-to-voltage converters convert current signals to voltage signals, and at least one lock-in amplifier and/or current or voltage amplifiers extract signals from noise. Depending on the target substance contained in the sample, the user uses appropriate formulas to calculate the amount of the substance in the sample using the extracted voltage signals with the data analysis system. In at least one embodiment, the calculations of the substance content are based on common chemical knowledge.

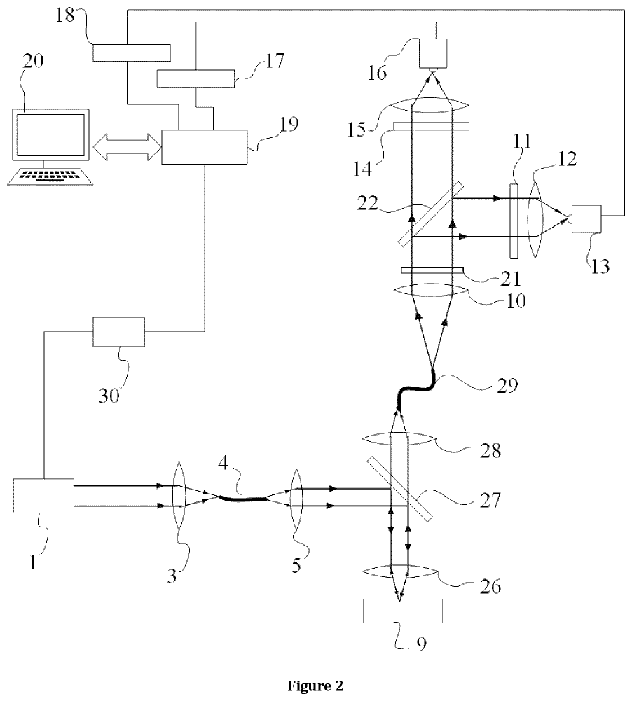

FIG. 2 depicts a schematic block diagram of a second embodiment of a system of the present disclosure. The embodiment in FIG. 2 includes an infrared light source 1 such as, for example, a 1064 nm continuous-wave Nd:YVO₄ laser. The initial light output from the light source 1 is generated using electronic gating. The light output may be modulated by periodic signals from the signal generator 30 connected to the light source 1 and then converged by lens 3 and coupled into the fiber optic cable 4, for example, a Thorlabs M38L01 patch cable. Lens 5 expands the initial light beam from the optical patch cable 4. The beam splitter 27 such as a Thorlabs DMLP1180 longpass dichroic mirror directs the incident beam towards lens 26, which focusses the incident beam on samples disposed on sample stage 9. Scattered light from the sample is collected by a scattering collection system which comprises lenses 26 and 28. The lens 28 couples the scattered light into fiber optic cable 29, such as a Thorlabs M59L02 patch cable. Then lens 10 expands the output light from the optical cable 29, and the rest of elements except the lock-in amplifier 19 work in the same way as they are in the first embodiment shown at FIG. 1. Although not shown in FIG. 2, the lenses 5, 26 and 28, and the beamsplitter 27, are integrated into a housing configured to be held by a user and moved adjacent the sample disposed on the sample stage 9 for measurements. The lock-in amplifier 19 in FIG. 2 uses the periodic signal from the signal generator 30 as the reference signal. The lock-in amplifier 19 may be connected, or otherwise intercommunicates, to a data analysis system 20 such as a computer, and the data analysis system outputs the content of the target substances present in the samples. The photon collection efficiency of the informative channel, in an embodiment, is 1:2.59×10¹¹, which indicates that one Raman photon carrying critical information is collected when every 2.59×10¹¹ incident photons interact with the sample. As an example of a method for real-time synchronous detection using this embodiment, the user powers the system, puts a sample on the sample stage 9 (or equivalent), moves the aforementioned housing adjacent the sample/sample stage 9, and controls the start and/or the end of measurements manually or automatically via the analysis system 20. The other steps of using the system are similar to the method shown in FIG. 1.

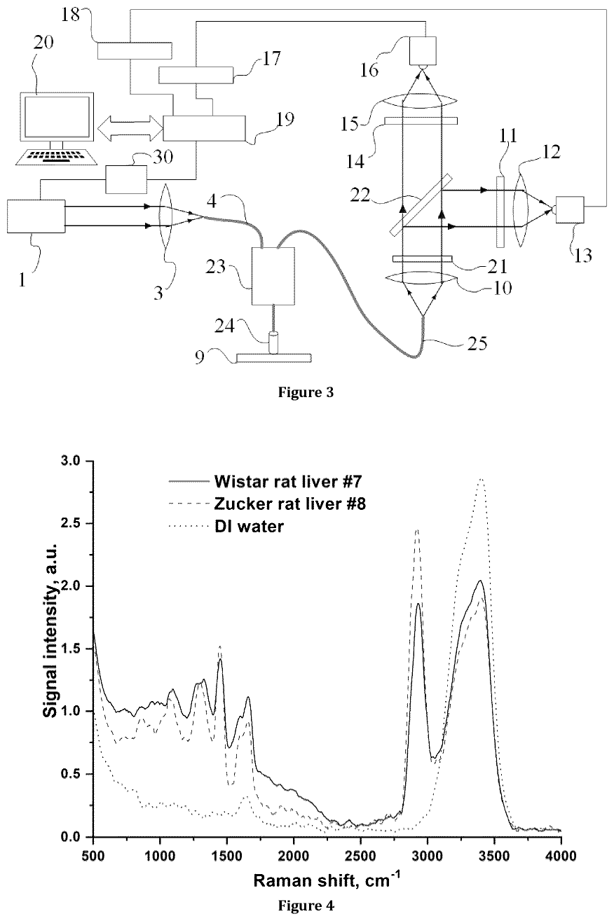

FIG. 3 depicts a schematic block diagram of a third embodiment of a system in accordance with the present disclosure. The system includes an infrared light source 1 such as, for example, a 1064 nm continuous-wave Nd:YVO₄ laser. The initial light beam from the light source 1 is generated using electronic gating. The light beam is modulated by periodic signals from the signal generator 30 connected to the light source 1 and then converged by lens 3 and coupled into the fiber optic cable 4 such as, for example, a Thorlabs M38L01 patch cable. The fiber optic cable 4 is connected to the optical coupler 23. The incident beam then goes through a fiber probe 24 and excites samples on sample stage 9. The samples are excited, and the scattered light is collected by the probe 24. The fiber probe 24 can be handheld. The coupler 23 couples the collected scattered light into optical cable 25. A scattering collection system comprising lenses 10, 12, and 15 collects the output light from the optical cable 25, then lens 10 expands the output light from the optical patch cable 25, and the rest of the elements work in the same way as described above in relation to the embodiment in FIG. 2. As an example of the method for a real-time synchronous detection using this embodiment, the user turns on power of the system, puts a sample on the sample stage (or equivalent), holds the fiber probe 24 adjacent the sample/sample holder 9, and controls the start and/or terminal of measurements manually or automatically via the analysis system 20. The other steps are similar to the method of using the system embodiment in FIG. 1.

### EXAMPLES

A phase scan technique was used to find the maximum signal for the sunflower oil, pork lard phantoms, duck fat phantoms and animal organ tissues of varying fat concentrations. A total of eight pork lard phantoms, eleven duck phantoms, twelve sunflower oil phantoms, and five animal organ samples were examined. The scan technique uses a step size of one degree from −180 degrees to +180 degrees. A triglyceride quantification colorimetric kit was used to determine the reference fat content in the animal organ tissues. The nominal fat content in the phantoms was calculated by the volume fractions of pork lard, duck fat, or sunflower oil in them.

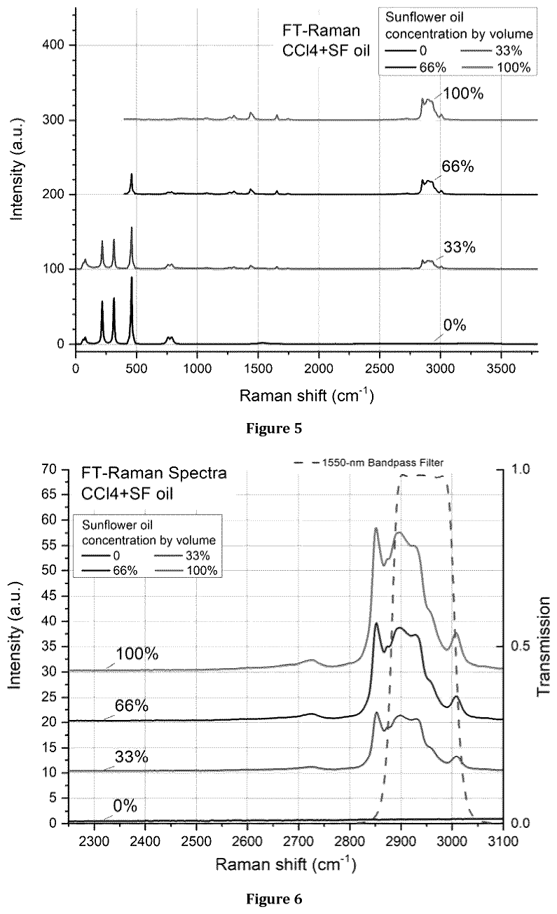

FIG. 4-6 shows the Raman spectra for fatty Zucker rat liver and normal Wistar rat livers between 500 and 4000 cm⁻¹ (1124-1734 nm) and the Raman spectrum of sunflower oil between 0 and 4210 cm⁻¹ (1024 nm and 1800 nm), respectively. Strong Raman characteristic peak at around 2900 cm⁻¹ (1550 nm) corresponds to the C—H bond in triglycerides, and it can be referred to as the fat peak. Deionized water has a strong peak at around 3250 cm⁻¹ but no peaks at 2900 cm⁻¹. The figures show the water peak at 3250 cm⁻¹ does not interfere with the fat peaks at 2900 cm⁻¹.

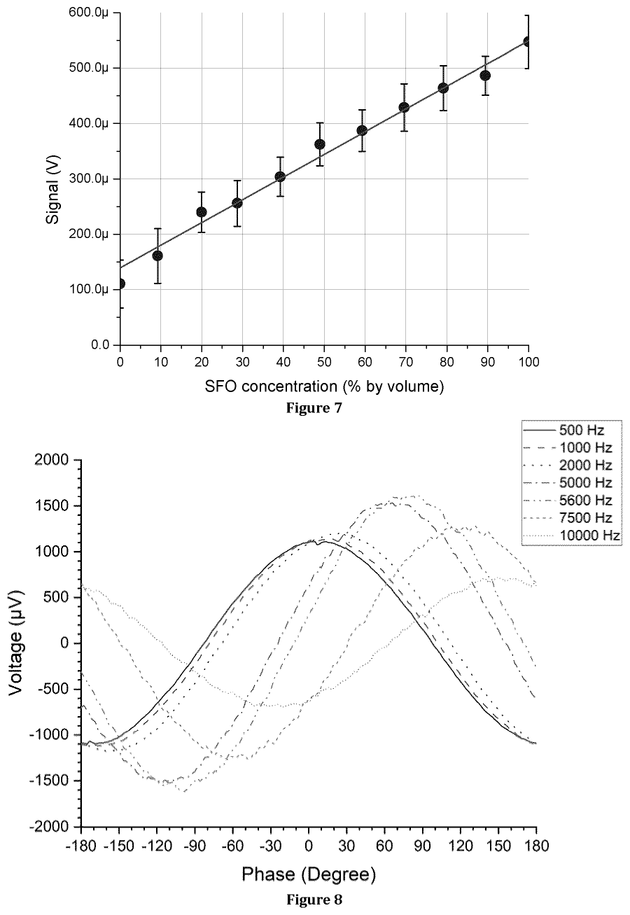

FIG. 7 shows the maximum signal voltage obtained with a miniaturized handheld probe in accordance with the present disclosure as a function of sunflower oil concentration in the sunflower oil phantoms within the sunflower oil concentration range of 0-100%. The voltage signals are measured at 3300 cm⁻¹ (1550 nm). These signals are linearly correlated to the sunflower oil content in the phantoms within the concentration range of 0-100%.

FIG. 8 shows phase scan plots obtained from the lock-in amplifier at different modulation (reference) frequencies. The informative voltage signal of the lock-in amplifier is measured when changing the phase difference between the reference periodic signal (provided by the optical modulator or the signal generator) and the informative signal. These are the results the measurements of Raman scattered photons (in this case, filtered by a 1550 nm band pass filter) from a pure pork lard phantom illuminated with 200 mW incident light at 1064 nm. Once the phase shift where the maximum voltage occurs is measured, the phase scan could be modified to scan just the range of interest to reduce scan time for repeated trials. In this case, the maximum voltage signal occurs at 60 degrees in phase at 5600 Hz modulation frequency.

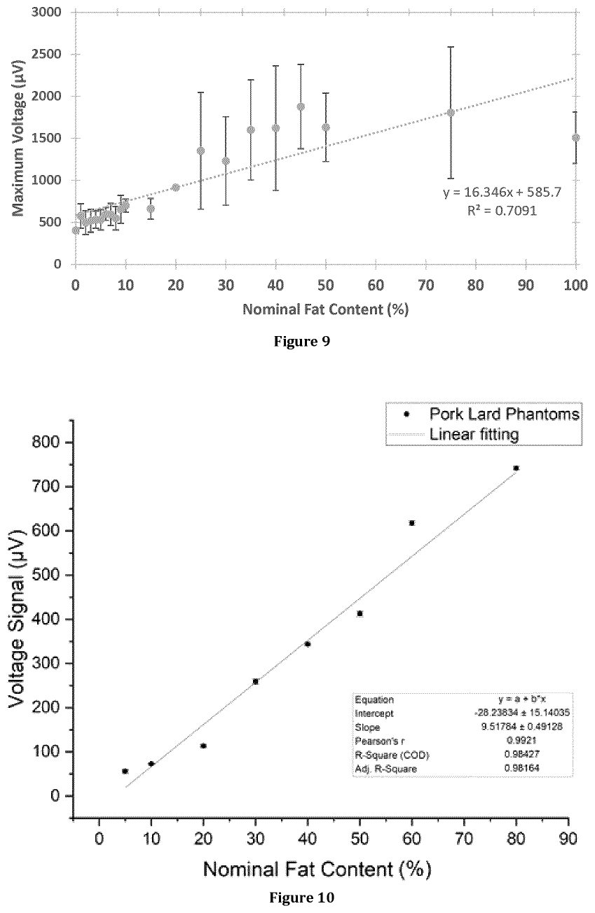

FIG. 9 shows the maximum signal voltage obtained with a tabletop probe in accordance with the present disclosure as a function of the sunflower oil concentration in the sunflower oil phantoms within the fat content range of 0-100%. The voltage signals are measured at 3300 cm⁻¹ (1550 nm). Each data point is a mean of 3 measurements at the same area of the phantom. The error bars represent one standard deviation. The incident light was modulated at 500 Hz by an optical modulator. The lock-in amplifier applied Is time constants (pre and post), and 10-time amplification. These results have a linear correlation between the voltage signals measured in accordance with the present disclosure and the nominal fat content in the phantoms within the fat range of 0-100%. The Pearson's correlation coefficient is equal to 0.71.

FIG. 10 shows the maximum voltage obtained with a tabletop probe in accordance with the present disclosure as a function of the fat content of the pork lard phantoms within the clinically relevant range. Each data point is a mean of 1000 measurements at the same area of the phantom. The error bars represent one standard deviation. The incident light was modulated at 500 Hz by an optical modulator. The lock-in amplifier applied 1s time constants (pre and post), 20 mV sensitivity, and 10-time amplification. The voltage signals were measured at 3300 cm⁻¹ (1550 nm). The Pearson's correlation coefficient is equal to 0.99.

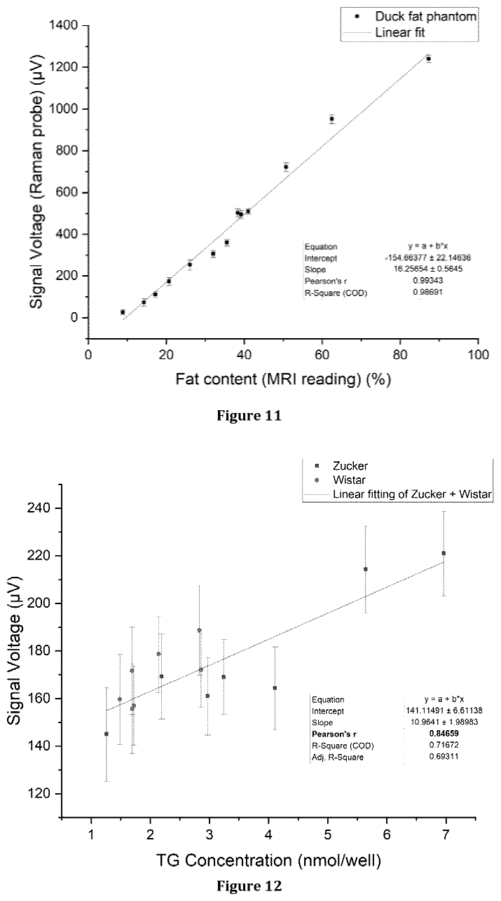

FIG. 11 shows the maximum voltage obtained with a handheld probe in accordance with the present disclosure as a function of the fat content of the duck fat phantoms for the clinically relevant range. Each data point is a mean of 1000 measurements at the same area of the phantom. The error bars represent one standard deviation. The lock-in frequency was 5600 Hz and was provided by a signal generator. The lock-in amplifier applies 1s time constants (pre and post), 20 mV sensitivity, and 10-time amplification. The Pearson's correlation coefficient is equal to 0.99.

FIGS. 9-11 show that embodiments of the present disclosure have a limit of detection of approximately 5%.

FIG. 12 provides a linear correlation between the maximum signal voltage obtained with a handheld probe in accordance with the present disclosure as a function of triglyceride (TG) concentration for ex-situ rodent livers (which is determined by a triglyceride quantification colorimetric kit). In the present embodiment, the approximation 1 nmol=2.45% fat content is used. Each data point is a mean of 1000 measurements at the same area of the phantom. The error bars represent one standard deviation. The Pearson's correlation coefficient is equal to 0.85. This confirms that embodiments of the present disclosure can provide accurate results on the fat content in animal ex-situ organ tissues.

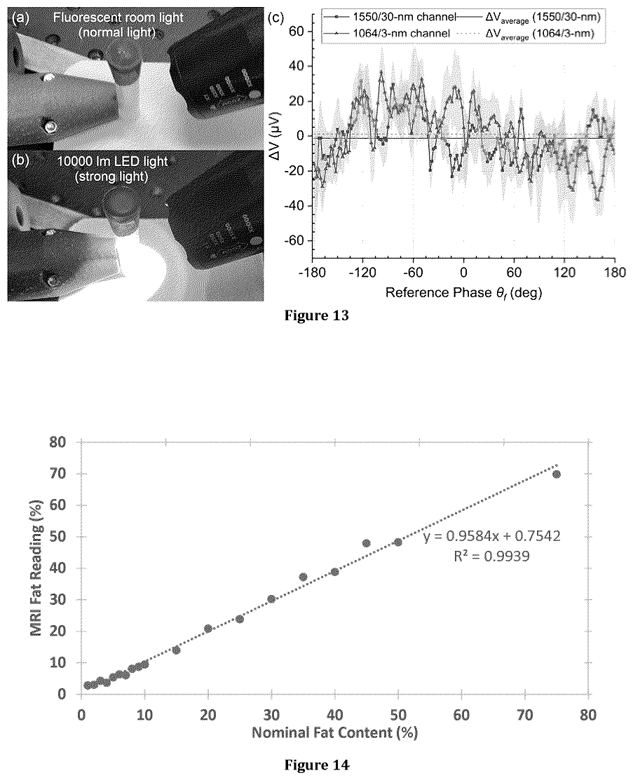

FIG. 13 provides voltage signal differences of fat content measurements of the duck fat phantoms using a handheld probe in accordance with the present disclosure at ambient LED light conditions and in the dark. Each data point is a mean of 1000 measurements at the same area of each phantom. The error bars represent one standard deviation. This confirms that embodiments of the present disclosure can provide statistically same results on the fat content in the phantoms at ambient LED light conditions and in the dark.

FIG. 14 provides fat concentration estimations of sunflower oil phantom using MRI. The MRI results are the most accurate estimate of the fat concentration of the phantoms. The Pearson's correlation coefficient is equal to 0.99.

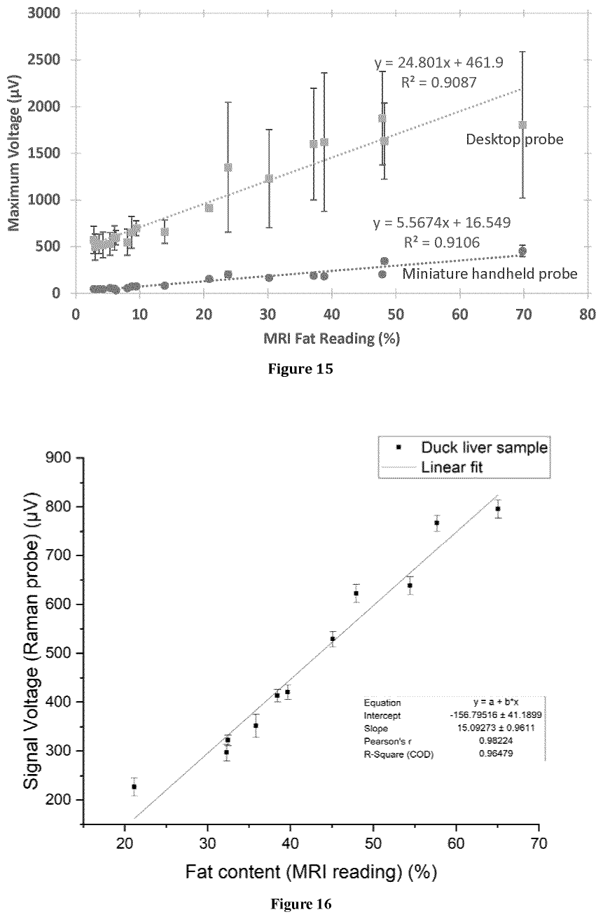

FIG. 15 represents the data collected with the tabletop probe (FIG. 1), and the miniature handheld probe (FIG. 3) as a function of the MRI fat estimates for the sunflower oil phantoms. It confirms the results obtained in accordance with the present disclosure and those obtained from MRI are comparable.

FIG. 16 represents the data collected using the handheld probe (FIG. 2), the handheld probe as a function of the MRI fat estimates for the fatty duck liver samples. It confirms the results obtained in accordance with the present disclosure and those obtained from MRI are comparable.

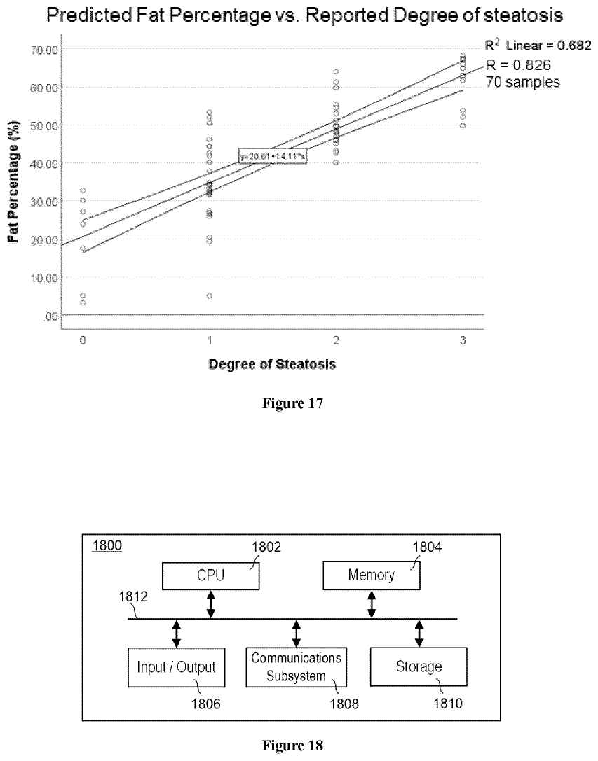

FIG. 17 represents the data collected with the miniature handheld probe (FIG. 3) as a function the degree of liver steatosis rated by pathologists [Dalhousie REB #2022-6318 approved]. It confirms the results obtained in accordance with the present disclosure and those obtained from pathologists' rating are comparable.

FIG. 18 is a block diagram of an example computerized device or system 1800 that may be used in implementing one or more aspects or components of an embodiment according to the present disclosure. For example, system 1800 may be used to implement a computing device or system, such as a controller, to be used with a device, system or method according to the present disclosure. In an embodiment, computerized system 1800 may be used to implement at least a portion of a data acquisition and/or analysis system, an optical system, data acquisition and analysis control hardware and/or software, process control software and/or hardware, a data processing and analysis system, and so on, according to the present disclosure.

Computerized system 1800 may include one or more of a computer processor device 1802, memory 1804, a mass storage device 1810, an input/output (I/O) interface 1806, and a communications subsystem 1808. A computer processor device may be any suitable device(s), and encompasses various devices, systems, and apparatus for processing data and instructions. These include, as examples only, one or more of a hardware processor, programmable processor, a computer, a system on a chip, and special purpose logic circuitry such as an ASIC (application-specific integrated circuit) and/or FPGA (field programmable gate array).

Memory 1804 may be configured to store computer readable instructions, that when executed by processor 1802, cause the performance of operations, including operations in accordance with the present disclosure.

One or more of the components or subsystems of computerized system 1800 may be interconnected by way of one or more buses 1812 or in any other suitable manner.

The bus 1812 may be one or more of any type of several bus architectures including a memory bus, storage bus, memory controller bus, peripheral bus, or the like. The processor 1802 may comprise any type of electronic data processor. The memory 1804 may comprise any type of system memory such as dynamic random access memory (DRAM), static random access memory (SRAM), synchronous DRAM (SDRAM), read-only memory (ROM), a combination thereof, or the like. In an embodiment, the memory may include ROM for use at boot-up, and DRAM for program and data storage for use while executing programs.

The mass storage device 1810 may comprise any type of storage device configured to store data, programs, and other information and to make the data, programs, and other information accessible via the bus 1812. The storage device may be adapted to store one or more databases and/or data repositories, each of which is generally an organized collection of data or other information stored and accessed electronically via a computer. The term database or repository may thus refer to a storage device comprising a database. The mass storage device 1810 may comprise one or more of a solid state drive, hard disk drive, a magnetic disk drive, an optical disk drive, or the like. In some embodiments, data, programs, or other information may be stored remotely, for example in the cloud. Computerized system 1800 may send or receive information to the remote storage in any suitable way, including via communications subsystem 1808 over a network or other data communication medium.

The I/O interface 1806 may provide interfaces for enabling wired and/or wireless communications between computerized system 1800 and one or more other devices or systems. Furthermore, additional or fewer interfaces may be utilized. For example, one or more serial interfaces such as Universal Serial Bus (USB) (not shown) may be provided. Further, system 1800 may comprise or be communicatively connectable to a display device, and/or speaker device, a microphone device, an input device such as a keyboard, pointer, mouse, touch screen display or any other type of input device.

Computerized system 1800 may be used to configure, operate, control, monitor, sense, and/or adjust devices, systems, and/or methods according to the present disclosure.

A communications subsystem 1808 may be provided for one or both of transmitting and receiving signals over any form or medium of digital data communication, including a communication network. Examples of communication networks include a local area network (LAN), a wide area network (WAN), telecommunications network, cellular network, an inter-network such as the Internet. and peer-to-peer networks such as ad hoc peer-to-peer networks. Communications subsystem 1808 may include any component or collection of components for enabling communications over one or more wired and wireless interfaces. These interfaces may include but are not limited to USB. Ethernet (e.g. IEEE 802.3), high-definition multimedia interface (HDMI). Firewire™ (e.g. IEEE 1374). Thunderbolt™. WiFi™ (e.g. IEEE 802.11). WiMAX (e.g. IEEE 802.16), Bluetooth™, or Near-field communications (NFC), as well as General Packet Radio Service (GPRS). Universal Mobile Telecommunications System (UMTS). Long-Term Evolution (LTE). LTE-A, 5G NR (New Radio), satellite communication protocols, and dedicated short range communication (DSRC). Communication subsystem 1808 may include one or more ports or other components (not shown) for one or more wired connections. Additionally or alternatively, communication subsystem 1808 may include one or more transmitters, receivers, and/or antenna elements (none of which are shown). Further, system 1800 may comprise clients and servers.

Computerized system 1800 of FIG. 18 is merely an example and is not meant to be limiting. Various embodiments may utilize some or all of the components shown or described. Some embodiments may use other components not shown or described but known to persons skilled in the art.

Logical operations of the various embodiments according to the present disclosure may be implemented as (i) a sequence of computer implemented steps, procedures, or operations running on a programmable circuit in a computer. (ii) a sequence of computer implemented operations, procedures, or steps running on a specific-use programmable circuit; and/or (iii) interconnected machine modules or program engines within the programmable circuits. The computerized device or system 1800 of FIG. 18 may practice all or part of the recited methods or operations, may be a part of systems according to the present disclosure, and/or may operate according to instructions in computer-readable storage media. Such logical operations may be implemented as modules configured to control a computer processor, such as processor 1802, to perform particular functions according to the programming of the module. In other words, a computer processor, such as processor 1802, may execute the instructions, steps, or operations according to the present disclosure, including of the one or more of the blocks or modules.

As will now be apparent to those of skill in the art, the present disclosure provides a novel system and method for analyzing biotic and abiotic system to determine fat concentrations. The system employs an ingenious combination of beam splitters/dichroic mirrors and optical pass filters and a lock-in or/and current or voltage amplification technique. The system is easy-to-use and can provide real-time and quantitative results about the content of molecules of end-users interests in the system.

Embodiments according to the present disclosure can be represented as a computer program product stored in a machine-readable medium (also referred to as a computer-readable medium, a processor-readable medium, or a computer usable medium having a computer-readable program code embodied therein). The machine-readable medium can be any suitable tangible, non-transitory medium, including magnetic, optical, or electrical storage medium including a diskette, compact disk read only memory (CD-ROM), memory device (volatile or non-volatile), or similar storage mechanism. The machine-readable medium can contain various sets of instructions, code sequences, configuration information, or other data, which, when executed, cause a processor to perform steps in a method according to an embodiment of the disclosure. Those of ordinary skill in the art will appreciate that other instructions and operations necessary to implement the described implementations can also be stored on the machine-readable medium. The instructions stored on the machine-readable medium can be executed by a processor or other suitable processing device and can interface with circuitry to perform the described tasks.

The structure, features, accessories, and/or alternatives of embodiments described and/or shown herein, including one or more aspects thereof, are intended to apply generally to all of the teachings of the present disclosure, including to all of the embodiments described and illustrated herein, insofar as they are compatible. Thus, the present disclosure includes embodiments having any combination or permutation of features of embodiments or aspects herein described.

In addition, the steps and the ordering of the steps of methods and data flows described and/or illustrated herein are not meant to be limiting. Methods and data flows comprising different steps, different number of steps, and/or different ordering of steps are also contemplated. Furthermore, although some steps are shown as being performed consecutively or concurrently, in other embodiments these steps may be performed concurrently or consecutively, respectively.

The above-described embodiments of the present invention are intended to be examples only. Alterations, modifications, and variations may be applied to the particular embodiment by those of skill in the art without departing from the scope of the invention, which is defined solely by the claims appended hereto.

1. An optical system for real-time synchronous detection of vibration and/or rotation modes in biotic and abiotic systems, the system comprising: a light source, an incident light path, a sample stage, a scattering collection system, single or multiple informative channels, single or multiple current-to-voltage converters, single or multiple current or voltage amplifiers, single or multiple lock-in amplifiers, photoelectric detectors, and a data analysis system, wherein a modulated light beam from the light source passes through the incident light path and excites a sample on the sample stage, and the collection system collects the scattered light emanating from the sample and focuses the light on the photoelectric detectors of the informative channels and a reference channel, and the single or multiple current-to-voltage converter, single or multiple current or voltage amplifier, and single or multiple lock-in amplifiers amplify the output electric signals of the photoelectric detectors, and the data analysis system analyzes amplified signals collected by the channels and outputs the substance content of the sample.

2. The optical system for real-time synchronous detection of vibration and/or rotation modes in biotic and abiotic systems of claim 1, wherein the data analysis system analyzes informative and reference signals extracted by the lock-in amplifiers and outputs the substance content of the sample.

3. The optical system for real-time synchronous detection of vibration and/or rotation modes in biotic and abiotic systems of claim 1, wherein the lock-in amplifiers extract informative and reference signals directly or not directly from the pre-amplified signals output from the transimpedance amplifiers and/or voltage amplifiers.

4. The optical system for real-time synchronous detection of vibration and/or rotation modes in biotic and abiotic systems of claim 1, wherein the system outputs a signal which is insensitive to ambient light conditions, including but not limited to LED light conditions.

5. The optical system for real-time synchronous detection of vibration and/or rotation modes in biotic and abiotic systems of claim 1, wherein the light source includes but is not limited to an infrared laser.

6. The optical system for real-time synchronous detection of vibration and/or rotation modes in biotic and abiotic systems of claim 1, wherein an initial light beam is modulated in periodic manner by an optical modulator or an electronic gating.

7. The optical system for real-time synchronous detection of vibration and/or rotation modes in biotic and abiotic systems of claim 1, wherein the incident light path guides the output light beam from the laser source, shapes the beam, and alters the incident angle of the beam to a sample.

8. The optical system for real-time synchronous detection of vibration and/or rotation modes in biotic and abiotic systems of claim 1, wherein the wavelength of the near infrared laser source and photoelectric detectors ranges from approximately 800 nm to approximately 1700 nm, and the optical elements are optimized for that wavelength range.

9. The optical system for real-time synchronous detection of vibration and/or rotation modes in biotic and abiotic systems of claim 1, wherein the light passes through a combination of any number of optical elements including but not limited to lenses, mirrors, filters, beam splitters, or optical fibres.

10. The optical system for real-time synchronous detection of vibration and/or rotation modes in biotic and abiotic systems of claim 1, wherein the photoelectric detectors, including but not limited to InGaAs photodiode detectors, convert optical signals into electric signals.

11. The optical system for real-time synchronous detection of vibration and/or rotation modes in biotic and abiotic systems of claim 1, wherein one or multiple transimpedance amplifiers amplify electric signals directly or indirectly from the photoelectric detectors, including but not limited to informative, reference, background or noise signals.

12. The optical system for real-time synchronous detection of vibration and/or rotation modes in biotic and abiotic systems of claim 1, wherein a combination of dichroic mirrors and optical pass filters selects specific optical wavelength bands of one or multiple informative channels.

13. The optical system for real-time synchronous detection of vibration and/or rotation modes in biotic and abiotic systems of claim 1, wherein the samples include but not limited to in-situ and ex-situ liver organs or tissues.

14. A method of detecting vibration and/or rotation modes of a biotic or abiotic sample, comprising: placing a sample to be tested onto a test stage, illuminating the sample with a light beam to excite the sample, collecting scattered light from the illuminated sample and selecting characteristic optical bands with beam splitters and optical pass filters, converting light signals to electric signals with at least one photoelectric detector, converting current signals to voltage signals with current-to-voltage converters, extracting signals from noise with at least one lock-in amplifier and/or voltage amplifier, and calculating the amount of the substance content in the sample using the analysing system and the extracted voltage signals.

15. The method of detecting vibration and/or rotation modes of a biotic or abiotic sample of claim 14, wherein the intensity of the extracted voltage signal of either channel represents the strength of characteristic vibration and/or rotation modes of the target substance.
